(19) United States
(12) Patent Application Publication
(10) Pub. No.: US 2026/0105750 A1
(43) Pub. Date: Apr. 16, 2026

(54) Methods and Systems for Detecting Anomalies in Utility Enclosures

(71) Applicant: CNIguard Ltd, Stanmore (GB)

(72) Inventors: James Darroch, Edinburgh (GB); Matt Daykin, Edinburgh (GB)

(73) Assignee: CNIguard Ltd, Stanmore, LN (GB)

(21) Appl. No.: 19/346,807

(22) Filed: Oct. 1, 2025

(30) Foreign Application Priority Data
Oct. 1, 2024 (GB) 2414437.0

(51) Int. Cl. G06V 20/52; G01N 33/00; G06V 10/26; G06V 10/98; G08C 17/02

(52) U.S. Cl. CPC G06V 20/52; G01N 33/0068; G06V 10/26; G06V 10/993; G08C 17/02

(57) ABSTRACT

Provided herein are systems, methods, and computer-readable media for monitoring utility enclosures. In one embodiment, a sensing device comprises a sensor—such as a visual or infrared camera—and a processing module configured to receive sensor output, process the data using stored algorithms to assess environmental conditions, and wirelessly send anomaly data (e.g., images, text descriptions, and metadata) to an analysis system. The analysis system includes a receiving module and an analysis module that processes the received data with algorithms to compare current sensor information to stored data and verify the anomaly's accuracy. In some embodiments, the processing algorithms operate with lower computational requirements than those in the analysis system. The invention further encompasses methods for detecting, processing, and transmitting sensor data and computer-readable media containing instructions to perform these tasks.

### CROSS REFERENCE

This application claims the benefit of priority of the United Kingdom Provisional Patent Application Serial No. 2414437.0 filed on Oct. 1, 2024, the disclosure of which is incorporated by reference in its entirety for all purposes.

### TECHNICAL FIELD

The present disclosure relates to methods and systems for detecting anomalies in utility enclosures, particularly, but not exclusively, to methods and systems for detecting anomalies in underground utility structures using visual and/or infrared images.

### BACKGROUND

All utility infrastructure assets degrade over time. In the electrical transmission and distribution industry, failure of underground cabling or other associated equipment may ultimately lead to serious (and potentially fatal) events concerning failure of enclosures, such as manholes. Pressure build-up due to chemical reactions can lead to fires if flammable gases are ignited by an electrical arc. This can be potentially followed by an explosion that displaces or launches the manhole cover into the air with devastating consequences to public safety and damage to vehicles and buildings.

Early detection of adverse conditions can ultimately prevent injury to the public, utility employees, firefighters, and also prevent damage to vehicles or buildings. Damage to the underground infrastructure results in power cuts and asset damage, while pre-event accumulations of carbon monoxide may lead to building explosions. Natural gas distribution network operators (DNOs) may also be affected, as network gas lines often run close to electrical conductors. Additionally, natural gas leaks may flow into electricity distribution vaults containing electrical conductors, which can in turn form a source of methane. Accurately assessing adverse conditions in these environments allows for early response from utility operatives to mitigate dangerous events by containing, repairing assets and scheduling maintenance thereof. Typical fault locations are at electrical cable splices, joints and leaking transformers containing combustible oil.

Current detection systems in utility enclosures can be inaccurate and slow, resulting in slow response times from utility operatives. This increases the likelihood of catastrophic events occurring.

There is therefore a requirement for an improved method of assessing anomalies in utility enclosures.

### SUMMARY

There is provided a system for monitoring one or more utility enclosures, the system comprising a sensing device, and an analysis system, wherein the sensing device comprises a sensor configured to detect information about a surrounding environment, and a processing module configured to receive sensor output from the sensor, process the sensor output using one or more algorithms stored on the processing module to determine whether an anomaly exists in the surrounding environment, and send data, based on output of the one or more algorithms, to the analysis system, and wherein the analysis system comprises a receiving module configured to receive the data from the sensing device, and an analysis module configured to process the received data using one or more algorithms stored on the analysis system to determine accuracy of the determined anomaly.

Optionally, the utility enclosure may be a maintenance hole, or a sewer hole, or a manhole. The utility enclosure may also be a vault, or a service box, or a mobile vehicle. The surrounding environment means the environment in which the sensor is located. For example, when the sensor is located within the utility enclosure, the surrounding environment is the environment within utility enclosure.

Optionally, the system may comprise a plurality of sensing devices. One or more of the plurality of sensing devices may be in communication with the analysis system. Optionally, each of the one or more sensing devices may comprise a plurality of sensors. The plurality of sensors and/or sensing devices may be configured to each detect different respective properties of the utility enclosure and/or each detect information about different respective areas of the surrounding environment.

Optionally, the processing module may be configured to perform the step of sending data to the analysis system when the anomaly is determined. This reduces the data/bandwidth burden by only sending data when anomalies may exist, and removes the need for constant data being sent to the analysis system. This also reduces the energy requirements of the sensing device.

Optionally, the data sent to the analysis system is the output of the one or more algorithms stored on the processing module. The result of the one or more algorithms may be a processed version of the data captured by the sensor. Optionally, it may be determined that an anomaly exists in the surrounding environment when an output of the one or more algorithms stored on the processing module satisfies a pre-determined condition, for example, exceeds a threshold, or falls within a range. The pre-determined condition may correspond to a property of the surrounding environment detected by the sensor.

Optionally, the detected anomaly may be one or more of: flooding within the utility enclosure; the utility enclosure being tampered with; electrical arcing; high carbon monoxide gas concentration level; high methane gas concentration level; stray voltage; rising rate of change of temperature; rising concentration of carbon monoxide; rising concentration of methane gas; areas of excessive heat; hot spots on equipment; surges in current in cables; changes in the overall structure of equipment; movement of equipment; high tertbutyl mercaptan (TBM) gas concentration level; high hydrogen sulphide gas concentration level; high ethylene gas concentration level; or high acetylene gas concentration level.

Optionally, the processing module may be configured to perform the step of sending data to the analysis system when the anomaly is determined.

Optionally, the processing module may be configured to perform the step of sending data to the analysis system at regular time intervals. These time intervals may be equal to or greater than a minute, an hour, a week, a day, a month, three months, six months, a year or multiple years.

Optionally, the processing module may be configured to send the data to the analysis system wirelessly. Optionally, the processing module may be in communication with the analysis system via a wireless connection such as a cellular connection or WiFi. Alternatively, the processing module may be in communication with the analysis system via a wired connection.

Optionally, the data may comprise an indication of the determined anomaly.

Optionally, the data may comprise output from the one or more sensors, and/or an indication of the one or more algorithms performed by the processing module. The data may be indicative of the surrounding environment.

Optionally, the sensor may be a camera and the output of the sensor is one or more images.

Optionally, the sensor may be a visual and/or infrared camera and the images may be visual and/or infrared images. Optionally, the sensor may be configured to detect a property of the environment surrounding the one or more sensing devices. The detected property may be one or more of acetylene gas concentration, arcing presence, contact temperature, carbon monoxide gas concentration, carbon monoxide gas concentration rate of change, current, ethylene gas concentration, flooding presence, humidity, hydrogen sulphide gas concentration, pressure, methane gas concentration, methane gas concentration rate of change, fault current, salinity concentration, stray voltage presence, temperature, temperature rate of change, tert-butyl mercaptan gas concentration, and/or water level.

Optionally, the analysis system may inform a user about an anomaly in the data when the analysis system determines that the anomaly detected by the sensing device is accurate.

There is further provided a system as described above, wherein the data comprises a text description, and/or one or more images, and optionally, tags assigned to the one or more images.

Optionally, the one or more algorithms stored on the processing module of the sensing device may have a lower computational requirement than the one or more algorithms stored on the analysis system.

Optionally, the one or more algorithms stored on the processing module may be configured to detect areas of high temperature and/or low temperature, bright spots, oil or gas leaks from pipes, movement of the sensor, structural damage, movement of objects within the scene/surrounding area, intruders and/or animals, flooding, and/or debris within the scene/surrounding area.

Optionally, the processing module of the sensing device may have a maximum processing speed of less than 5 Ghz, or preferably, less than 2 GHz.

Optionally, the processing module of the sensing device may have a maximum processing speed of 1.8 GHz.

Optionally, the processing module of the sensing device may have a maximum SDRAM memory of less than 50 GB, preferably less than 10 GB.

Optionally, the processing module of the sensing device may have a maximum SDRAM memory of 8 GB.

Optionally, the processing module has a relatively low computational power compared to the computational power of the analysis system. For example, the processing module may have a maximum computational power of less than 50% of the maximum computational power of the analysis system. This permits the use of a small processing module that requires minimal power to be used in the sensing device. The processing module may perform analysis requiring low-computational requirements before sending the data to the analysis system which can perform analysis requiring high-computational requirements, resulting in a more efficient system.

Optionally, the one or more algorithms stored on the processing module may comprise an algorithm operable on sensor output to determine if the sensor output is of low quality, and/or improve the quality of the sensor output.

Optionally, one of the one or more algorithms stored on the processing module may determine if movement of the scene and/or sensing device has occurred. A portion of the surrounding area viewed by the camera of the sensor is referred to as the scene.

Optionally, an output determined to be of low quality may comprise a blurry image, corrupt data, background noise, vertical bars in the image, and/or corrupt/erroneous pixels.

Optionally, improving the quality of the sensor output may comprise repairing minor errors in the images such as erroneous pixels, enhancing dark areas within the images, filtering the images to remove undesirable artifacts or contamination, and/or aligning and overlaying the original image with the processed image.

Optionally, the one or more algorithms stored on the analysis system may comprise an algorithm operable to determine differences between the received data and stored data, wherein the stored data is data stored by the analysis system and received at a time before the received data.

Optionally, the stored data may be data manually input into the analysis system by a user, and/or the stored data may be a range of target values. Optionally, the stored data may be received from the sensing device at a time previous to the received data.

Optionally, the processing module may be configured to perform the step of sending data to the analysis system at regular time intervals as well as also sending data to the analysis system when anomalies are detected by the processing module. This ensures that the stored data on the analysis system is regularly updated, as well as ensuring that the processing module is not malfunctioning and failing to send data when anomalies are present.

Optionally, the one or more algorithms stored on the analysis system may comprise computationally demanding algorithms such as image segmentation, or algorithms utilising stored/historical data.

Optionally, the stored data may be indicative of the environment surrounding the sensing device at a time previous to the received data. The time may be equal to or greater than a minute, an hour, a week, a day, a month, three months, six months, a year or multiple years.

There is further provided a sensing device for a utility enclosure, the sensing device comprising a sensor configured to detect information about a surrounding environment, and a processing module configured to receive sensor output from the sensor, process the sensor output using one or more algorithms stored on the processing module to determine whether an anomaly exists in the surrounding environment, and send data, based on output of the one or more algorithms, to an analysis system.

Optionally, the processing module may be configured to perform the step of sending data to the analysis system when the anomaly is determined. This reduces the data/bandwidth burden by only sending data when anomalies may exist. This removes the need for constant data being sent to the analysis system. This also reduces the energy requirements of the sensing device.

Optionally, the processing module may be configured to perform the step of sending data to the analysis system at regular time intervals. These time intervals may be any time interval between 1 second and 10 years.

Optionally, the output of the one or more algorithms stored on the processing module may be the result of the one or more algorithms. Optionally, it may be determined that an anomaly exists in the surrounding environment when an output of the one or more algorithms stored on the processing module satisfies a pre-determined condition, for example, exceeds a threshold, or falls within a range. The pre-determined condition may correspond to a property of the surrounding environment detected by the sensor.

Optionally, the sensor may be a camera and the output of the sensor may be one or more images, and the one or more algorithms stored on the processing module may comprise an algorithm operable on each of the sensor output image(s) to determine if each image is of low quality, and/or improve the quality of the sensor output.

Optionally, the sensor may be a visual and/or infrared camera and the images may be visual and/or infrared images. Optionally, the sensor may be configured to detect a property of the environment surrounding the one or more sensing devices. The detected property may be one or more of acetylene gas concentration, arcing presence, contact temperature, carbon monoxide gas concentration, carbon monoxide gas concentration rate of change, current, ethylene gas concentration, flooding presence, humidity, hydrogen sulphide gas concentration, pressure, methane gas concentration, methane gas concentration rate of change, fault current, salinity concentration, stray voltage presence, temperature, temperature rate of change, tert-butyl mercaptan gas concentration, and/or water level.

Optionally, the one or more algorithms stored on the processing module may comprise algorithms that are computationally less-demanding than the one or more algorithms stored on the analysis system. For example, the processing module may have a maximum computational power of less than 50% of the maximum computational power of the analysis system. This permits the use of a small processing module that requires minimal power to be used in the sensing device. The processing module may perform analysis requiring low-computational requirements before sending the data to the analysis system which can perform analysis requiring high-computational requirements, resulting in a more efficient system.

Optionally, one of the one or more algorithms stored on the processing module may determine if movement of the scene and/or sensing device has occurred.

Optionally, an output determined to be of low quality may comprise a blurry image, corrupt data, background noise, vertical bars in the image, and/or corrupt/erroneous pixels.

Optionally, improving the quality of the sensor output may comprise repairing minor errors in the images such as erroneous pixels.

Optionally, the processing module of the sensing device may have a maximum processing speed of less than 5 Ghz, or preferably, less than 2 GHz.

Optionally, the processing module of the sensing device may have a maximum processing speed of 1.8 GHz.

Optionally, the processing module of the sensing device may have a maximum SDRAM memory of less than 50 GB, preferably less than 10 GB.

Optionally, the processing module of the sensing device may have a maximum SDRAM memory of 8 GB.

There is further provided an analysis system for monitoring utility enclosures, the analysis system comprising a receiving module configured to receive data from a sensing device located at a utility enclosure, and an analysis module configured to process the received data using one or more algorithms stored on the analysis system to determine an accuracy of an anomaly of the utility enclosure indicated by the received data.

Optionally, the data may comprise an indication of the determined anomaly.

Optionally, the data may comprise a detected property of the surrounding environment detected by the one or more sensing devices. The data may be indicative of the surrounding environment detected by the one or more sensing devices and may be one or more of acetylene gas concentration, arcing presence, contact temperature, carbon monoxide gas concentration, carbon monoxide gas concentration rate of change, current, ethylene gas concentration, flooding presence, humidity, hydrogen sulphide gas concentration, pressure, methane gas concentration, methane gas concentration rate of change, fault current, salinity concentration, stray voltage presence, temperature, temperature rate of change, tert-butyl mercaptan gas concentration, and/or water level.

Optionally, the received data may comprise one or more images and the one or more algorithms stored on the analysis system may comprise image segmentation.

Optionally, the one or more algorithms stored on the analysis system may comprise computationally demanding algorithms such as image segmentation, or algorithms utilising stored/historical data.

Optionally, the one or more algorithms stored on the analysis system may comprise an algorithm operable to determine differences between the received data and stored data, wherein the stored data is data stored by the analysis system and received at a time before the received data.

Optionally, the stored data may be data manually input into the analysis system by a user, and/or the stored data may be a range of target values. Optionally, the stored data may be received from the sensing device at a time previous to the received data.

Optionally, the stored data may be indicative of the environment surrounding the sensing device at a time previous to the received data. The time may be equal to or greater than a minute, an hour, a week, a day, a month, three months, six months, a year, or multiple years.

There is provided a method of monitoring a utility enclosure, the method comprising detecting, within the utility enclosure, information about the utility enclosure, processing, within the utility enclosure, the detected information using one or more algorithms stored on a processing module to determine whether an anomaly exists in the environment, and sending data based on output of the one or more algorithms stored on the processing module to an analysis system outside of the utility enclosure, receiving the data at the analysis system, and further processing the received data, by the analysis system, using one or more algorithms stored on the analysis system to determine accuracy of the determined anomaly.

Optionally, the method may be for monitoring one or more utility enclosures.

Optionally, the one or more utility enclosures may be maintenance holes, or sewer holes, or manholes. The one or more utility enclosures may also be vaults, or service boxes, or mobile vehicles.

Optionally, the data sent to the analysis system is the output of the one or more algorithms stored on the processing module. Optionally, it may be determined that an anomaly exists in the surrounding environment when an output of the one or more algorithms stored on the processing module satisfies a pre-determined condition, for example, exceeds a threshold, or falls within a range. The pre-determined condition may correspond to a property of the surrounding environment detected by the sensor.

Optionally, the analysis system may be remote from the utility enclosure.

Optionally, the processing module may be configured to send the data to the analysis system wirelessly. Optionally, the processing module may be in communication with the analysis system via a wireless connection such as a cellular connection or WiFi. Alternatively, the processing module may be in communication with the analysis system via a wired connection.

Optionally, the step of detecting may be via one or more sensors located at the one or more utility enclosures.

Optionally, the step of sending data to the analysis system may be performed when the anomaly is determined. This reduces the data/bandwidth burden by only sending data when anomalies may exist. This removes the need for constant data being sent to the analysis system. This also reduces the energy requirements of the sensing device.

Optionally, the one or more algorithms performed within the utility enclosure may have a lower computational requirement than the one or more algorithms performed by the analysis system.

Optionally, the one or more algorithms performed within the utility enclosure may comprise an algorithm operable on detected information to determine if the detected information is of low quality, and/or improve the quality of the detected information.

Optionally, one of the one or more algorithms stored on the processing module may determine if movement of the scene and/or sensing device has occurred.

Optionally, an output determined to be of low quality may comprise a blurry image, corrupt data, background noise, vertical bars in the image, and/or corrupt/erroneous pixels.

Optionally, the one or more algorithms performed by the analysis system may comprise an algorithm operable to determine differences between the received data and stored data, wherein the stored data is data stored by the analysis system and received at a time before the received data.

Optionally, the one or more algorithms stored on the analysis system may comprise computationally demanding algorithms such as image segmentation, or algorithms utilising stored/historical data.

Optionally, the stored data may be indicative of the environment surrounding the sensing device at a time previous to the received data. The time may be equal to or greater than a minute, an hour, a week, a day, a month, three months, six months, a year, or multiple years.

There is further provided a computer-readable storage medium storing instructions adapted to carry out the method described above of monitoring a utility enclosure.

There is further provided a computer-implemented method of detecting a hot spot in a utility enclosure, the method may comprise receiving an infrared image of the utility enclosure, wherein the infrared image may comprise a plurality of pixels. The method may further comprise the steps of determining a temperature of each of the plurality of pixels within the infrared image, calculating a range of the determined temperatures, wherein the range of the determined temperatures is a difference between a highest temperature of the determined temperatures and a lowest temperature of the determined temperatures, assessing whether the range of the determined temperatures is above a threshold range value, when the range of the determined temperatures is above the threshold range value, determining a percentage of the plurality of pixels with temperatures above a threshold temperature value, wherein the threshold temperature value is a temperature between the highest temperature and the lowest temperature, and detecting the hot spot when the percentage of the plurality of pixels with temperatures above the threshold temperature value is below a threshold percentage of the plurality pixels.

The hot spot may be an area of excessive heat within the utility enclosure.

The determined temperature of each of the plurality of pixels may be a temperature of the environment represented by the pixel. For example, each pixel of the infrared image may be representative of the temperature of the environment at the location represented by the pixel in the infrared image.

Each of the plurality of pixels may have a corresponding determined temperature. Calculating a range of the determined temperatures of the plurality of pixels may require determining a temperature of each of the plurality of pixels, and determining the difference in temperature between the highest determined temperature and the lowest determined temperature.

Optionally, the plurality of determined temperatures may be ranked in order from highest temperature to lowest temperature.

The threshold temperature value may be determined from the plurality of determined temperatures. The threshold temperature may be at a predetermined position within the range of the determined temperatures. For example, the threshold temperature value may be at a predetermined position which equates to 80% the way through the range of predetermined temperatures. For example, if the determined temperatures range from 60 degrees Celsius to 70 degrees Celsius, then the range is equivalent to 10 degrees Celsius (i.e., 70 degrees Celsius−60 degrees Celsius). Therefore, the threshold temperature value would be 60 degrees Celsius+80% of the range (i.e., 8 degrees Celsius)=68 degrees Celsius. Likewise, if the determined temperatures range from 63 degrees Celsius to 70 degrees Celsius, the threshold temperature value would be 63 degrees Celsius+80% of the range (i.e., 5.6 degrees Celsius)=68.6 degrees Celsius.

Optionally, when the range of the determined temperatures is below the threshold range value, no hot spot is detected.

Optionally, the method may further comprise creating, from the infrared image, a new image comprising the pixels with determined temperatures above the threshold temperature value, when more than or equal to one and less than all of the determined temperatures are above the threshold temperature value.

Optionally, the new image may only be created when the percentage of the plurality of pixels with temperatures above the threshold temperature value is below a threshold percentage of the plurality pixels. The new image may only be created when a hot spot has been detected.

The new image may represent the pixels equal to or greater than the threshold temperature value in one colour (for example, white) and may represent the pixels below the threshold temperature value in another colour (for example, black). The pixels represented in the new image may all correspond in location to their respective pixels in the infrared image. The new image may highlight the specific locations of the pixels with determined temperatures above the threshold temperature value, assisting a user in visualizing the location of the determined hot spot.

Optionally, the threshold range value may be between 1 degree Celsius and 50 degrees Celsius. The threshold range value may be between 2 degrees Celsius and 15 degrees Celsius. The threshold range value may be between 4 degrees Celsius and 8 degrees Celsius. The threshold range value may be between 5 degrees Celsius and 6 degrees Celsius.

Optionally, the threshold range value may be any value selected by a user of the method or may be any value selected by the method.

Optionally, the threshold percentage of the plurality of pixels may be between 0.5% and 2%. The threshold percentage of the plurality of pixels may be greater than 0.1%, or 0.5%. The threshold percentage of the plurality of pixels may be less than 5%, or 2%. The threshold percentage of the plurality of pixels may be 1%. Optionally, the threshold percentage of the plurality of pixels may be greater than 0% and less than 100%.

Optionally, the threshold percentage may be any percentage selected by a user of the method or may be any percentage selected by the method.

Optionally, the method may further comprise one or more of the steps of determining if the received infrared image is useable, and/or correcting errors within the received infrared image (for example, erroneous pixels), and/or enhancing dark areas within the received infrared image, and/or filtering the received infrared image to remove artifacts or contamination, and/or aligning and overlaying one or more of a received infrared image, visual image and/or processed image.

There is further provided a method of detecting a cold spot. The method of detecting a cold spot is as described above for a hot spot, except that a cold spot is detected when the percentage of the plurality of pixels with temperatures below the threshold temperature value is below a threshold percentage of the plurality pixels. The threshold temperature value may be at a predetermined position which equates to 20% the way through the range of predetermined temperatures.

There is further provided a computer-implemented method of detecting a structural change in a utility enclosure. The method may comprise receiving a first image of the utility enclosure, dividing the first image into a first plurality of segments using an image segmentation process, storing the first image comprising the plurality of segments, receiving a second image of the utility enclosure and dividing the second image into a second plurality of segments using the image segmentation process, comparing each segment of the second image with a corresponding one of the segments of the first image, wherein comparing comprises assessing for structural similarity, assigning, based on the assessment of structural similarity, a structural similarity score to each comparison, and detecting a structural change in the utility enclosure when the structural similarity score of one or more of the comparisons is below a threshold structural similarity score.

Optionally, the segmentation process may comprise segmenting the first image and/or second image into a plurality of equally sized segments, wherein each of the plurality of segments comprises the same number of pixels. The size of the segments may be selected by a user of the method or may be selected by the method. The method may select the most appropriate size of the plurality of segments for the specific dimensions of the first image and/or second image.

If, due to the selected size of the segments, the first image and/or second image cannot be perfectly divided up into equally sized segments, the method may discard pixels at one or more of the extremities of the first image and/or second image. For example, if an image consists of 1024×1024 pixels, and 200×200 pixel segments are selected, the method may discard the 24 columns of pixels on the right of the image and the 24 rows of pixels on the bottom of the image, permitting the first image and/or second image to be segmented into 25 200×200 pixel segments. On the other hand, if 256×256 pixel segments are selected for an image consisting of 1024×1024 pixels, the first image and/or second image is segmented into 16 equally sized 256×256 pixel segments without the need to discard any pixels from the first image and/or second image.

Due to the use of the image segmentation process for both the first and second images, each of the plurality of segments of the first image correspond in location with a respective one of the plurality of segments of the second image. The second image is segmented using the same image segmentation process as the first image such that the segments in both the first image and second image correspond with each other.

Optionally, the method may further comprise the step of forming a colour-coded image from the first image or the second image, wherein the colour-coded image comprises the first or second plurality of segments, and wherein each of the plurality of segments may be assigned a colour code corresponding to the structural similarity score of the respective comparison. The colour coding may emphasise the segments of the one or more colour-coded image(s) where changes in structure are detected.

Optionally, each of the plurality of segments may be assigned a numerical value corresponding to the structural similarity score of the comparison. Optionally, the numerical value ranges from 0 to 1, whereby 0 indicates very low structural similarity between the two compared segments, and 1 indicated a very high structural similarity between the two compared segments, i.e., the two segments are identical.

Optionally, the first image and second image may be images of the same environment, whereby the first image and second image are taken using a camera, and wherein the same camera is used to take both the first image and the second image. The camera may be located in the same position when taking both the first image and the second image.

Optionally, each of the plurality of segments comprises a common number of pixels. For example, each of the plurality of segments in the first image and/or second image may comprise 200×200 pixels (i.e., 40,000 pixels).

Optionally, the one or more formed colour-coded images may be output to the user. The formed colour-coded images may permit the user to visualize the location of the determined structural change.

Optionally, the method may further comprise one or more of the steps of determining if the received infrared image is useable, and/or correcting errors within the received infrared image (for example, erroneous pixels), and/or enhancing dark areas within the received infrared image, and/or filtering the received infrared image to remove artifacts or contamination, and/or aligning and overlaying one or more of a received infrared image, visual image and/or processed image.

There is further provided a computer-implemented method of detecting a hazardous condition in a utility enclosure, the method may comprise receiving a primary reading comprising a concentration of a first gas in the utility enclosure or a stray voltage, receiving one or more secondary readings taken in the utility enclosure, and detecting a hazardous condition based on the primary reading and the one or more secondary readings.

Optionally, the hazardous condition may be a dangerous event, such as a fire or explosion. The hazardous condition may be a condition that is indicative of an increased risk of a dangerous event within the utility enclosure, for example, an increased risk of a fire or explosion. The method may be configured to detect a potentially dangerous event in the utility enclosure, or an ongoing dangerous event. The method may be configured to identify hazardous conditions within the utility enclosure that may be indicative of a future dangerous event.

Optionally, one or more of the primary reading and/or the one or more secondary readings may be measurements (e.g., a direct measurement from a sensor), or may be an output result from one or more algorithms configured to process measurements of one or more properties of the utility enclosure. The one or more of the primary reading and/or the secondary reading(s) may be a positive identification of an event occurring in the utility enclosure, for example, a hot spot, and/or a gas leak, and/or a cold spot, and/or excessive heat, and/or excessive cold, and/or structural damage, and/or structural movement within the utility enclosure, and/or flooding, and/or debris within the utility enclosure, and/or intruders detected in the utility enclosure. The primary readings may be stray voltage, methane concentration, or carbon monoxide concentration. The secondary readings may be hot spot presence, methane concentration, carbon monoxide concentration, tert-butyl mercaptan concentration, temperature and/or stray voltage.

Optionally, each of the primary reading and the one or more secondary readings are of a different respective property of the utility enclosure.

Optionally, the method may further comprise determining a danger value based on the primary reading and the one or more secondary readings, and detecting a hazardous condition when the danger value is above a threshold danger value.

Optionally, the first gas may be methane.

Optionally, the first gas may be carbon monoxide.

Optionally, one of the one or more secondary readings may be a hot spot as determined by the computer-implemented method of detecting a hot spot.

Optionally, one of the one or more secondary readings may be an area of excessive heat.

Optionally, when the first gas is methane, one of the one or more secondary readings may be a concentration of carbon monoxide. The combination of carbon monoxide concentration and methane concentration to determine the presence of a hazardous event can improve the accuracy of the determination and reduce false alarms, for example due to vehicles present near the utility enclosure.

Optionally, one of the one or more secondary readings may be a concentration of methane.

Optionally, one of the one or more secondary readings may be a rate of change of concentration of methane. Optionally, one of the one or more secondary readings may be a rate of change of concentration of carbon monoxide.

Optionally, one of the one or more secondary readings may be a temperature.

Optionally, one of the one or more secondary readings may be a positive detection of tert-butyl mercaptan.

Optionally, a danger score may be assigned to one or more of the primary reading and to the one or more secondary readings. The danger value may be determined from the one or more danger scores.

Optionally, each of the primary reading and the one or more secondary readings may be assigned a separate danger score. Each of the danger scores assigned to the one or more of the primary reading and to the one or more secondary readings may have a different value.

Optionally, the danger value may be determined by adding together the individual danger scores assigned to each of the one or more of the primary reading and to the one or more secondary readings.

Optionally, when the first gas is methane, if excessive heat and/or a hot spot are detected as part of the one or more secondary readings, the danger score associated with the detection of a hot spot may be set to a maximum possible value, for example, when the range of danger scores is 0 to 1, the danger score for the detection of a hot spot may be 1.

Optionally, when the first gas is methane, if two consecutive methane readings are above 9999 ppm, or, above a saturation threshold of the methane sensor, the danger score associated with the methane reading may be set to a maximum possible value, for example, when the range of danger scores is 0 to 1, the danger score for the detection of a hot spot may be 1.

Optionally, when the first gas is methane and the methane reading is below 9999 ppm or below a saturation threshold of the methane sensor, and when the methane reading is above a methane alarm threshold, the danger score for the methane reading may be increased, for example, it may be increased by an amount correlating with a difference between the level of methane detected and the methane alarm threshold. For example, when the range of danger scores is 0 to 1, a danger score of between 0 and 0.5 may be assigned to the methane reading, the danger score increasing as the difference between the methane reading and the methane alarm threshold increases. The danger score may increase linearly as the difference between the methane reading and the methane alarm threshold increases.

Optionally, when the first gas is methane, if a reading of the concentration of methane is greater than a previous reading of the concentration of methane, a danger score value correlating to the increase in methane concentration may be assigned to the methane reading.

Optionally, when the first gas is methane, if tert-butyl mercaptan is detected as part of the one or more secondary readings, a danger score below the maximum danger score value, for example a tenth of the maximum score, may be assigned to the tert-butyl mercaptan reading. For example, when the range of danger scores is 0 to 1, a danger score of 0.1 may be assigned to the tert-butyl mercaptan reading.

Optionally, when the first gas is methane, if the methane reading is greater than or equal to 5000 ppm and optionally also greater than or equal to the methane alarm threshold and carbon monoxide is detected as part of the one or more secondary readings at a level above a baseline expected value, the danger score for the carbon monoxide reading may be increased, for example, it may be increased by an amount correlating with the level of carbon monoxide detected, optionally by an amount such as 20% of the maximum score. For example, when the range of danger scores is 0 to 1, or 0.2 may be added for every standard deviation which the carbon monoxide reading is above the baseline expected value.

A baseline for a reading may be determined by measuring the reading a plurality of times and determining an average and one or more standard deviations. A threshold for a reading can be determined as a number of standard deviations away from the baseline. The use of baselining improves the accuracy of the determination of a hazardous condition.

Optionally, when the first gas is carbon monoxide, if methane is detected as part of the one or more secondary readings and the detected level of methane is below a threshold value, for example 500 ppm, a low danger hazardous condition may be detected.

Optionally, when the first gas is carbon monoxide, if methane is detected as part of the one or more secondary readings and the detected level of methane is below a threshold value, for example 500 ppm, further assessment of the danger scores of the other readings may be terminated and a low danger hazardous condition may be detected. The combination of readings of methane and CO concentration helps to filter out false alarms caused by vehicle emissions while still detecting true alarms causes by serious situations like burning.

Optionally, when the first gas is carbon monoxide, if excessive heat and/or a hot spot are detected as part of the one or more secondary readings, the danger score associated with the detection of a hot spot/excessive heat may be set to a maximum possible value, for example, when the range of danger scores is 0 to 1, the danger score for the detection of a hot spot may be 1.

Optionally, when the first gas is carbon monoxide, and temperature is one of the secondary readings, when the temperature is higher than a threshold, for example, more than two standard deviations above a baseline expected temperature, then the danger score associated with the detection of the temperature may be set to a maximum possible value, for example, when the range of danger scores is 0 to 1, the danger score for the detection of a hot spot may be 1.

Optionally, when the first gas is carbon monoxide and the detected level of carbon monoxide has increased compared to a previous carbon monoxide reading, the danger score for the carbon monoxide reading may be increased, for example, it may be increased by an amount correlating with the increased level of carbon monoxide detected. For example, when the range of danger scores is 0 to 1, a danger score of 0.1 may be added for every 100 ppm increase in the carbon monoxide reading compared to the previous carbon monoxide reading.

Optionally, when the first gas is carbon monoxide and one of the one or more secondary readings is methane, the danger score for the methane reading may be increased, for example, it may be increased by an amount correlating with the increased level of methane detected compared to a previous methane reading. For example, when the range of danger scores is 0 to 1, a danger score of 0.05 may be added for every 1000 ppm increase in the methane reading compared to the previous methane reading.

Optionally, the danger value may be determined by adding the one or more danger scores. A danger value of greater than or equal to a high danger threshold value may be deemed as a high danger hazardous condition. The high danger threshold value may be equal to a maximum value for a danger score. A danger value of less than a medium danger threshold value may be deemed as a low danger hazardous condition. A danger value greater than or equal to the medium danger threshold value and less than the high danger threshold value may be deemed as a medium danger hazardous condition.

For example, when the range of danger scores is 0 to 1, a medium danger threshold value of 0.5 and a high danger threshold value of 1 may be used to determine whether a hazardous condition has been detected. In this example, a danger value of greater than or equal to 1 would reach/exceed the high danger threshold value, indicating a high danger hazardous condition. A danger value greater than or equal to 0.5 and less than 1 would reach/exceed the medium danger threshold value, indicating a medium danger hazardous condition. A danger value of less than 0.5 would not reach the medium danger threshold value, indicating a low danger hazardous condition.

Optionally, if any of the primary reading or one or more of the secondary readings is assigned a danger score with the maximum possible value, further assessment of the danger scores of the other readings may be terminated and the danger value may be output at the high danger threshold value, indicating a high danger hazardous condition.

Optionally, the method may further comprise outputting an alarm, wherein the alarm is indicative of the determined level of danger. The alarm may only be output when the determined level of danger is a high danger hazardous condition and/or a medium danger hazardous condition.

Optionally, the danger scores for any of the primary reading and the one or more secondary readings may be any other suitable value relative to the range of the danger scores. Optionally, the danger values deemed as low, medium and high danger hazardous conditions may be any other suitable value.

Further features and advantages of the aspects of the present disclosure will become apparent from the claims and the following description.

### BRIEF DESCRIPTION OF DRAWINGS

Embodiments of the present disclosure will now be described by way of example only, with reference to the following diagrams, in which:

FIG. 1 shows a schematic diagram of a system for monitoring utility enclosures;

FIG. 2 shows a schematic diagram of an alternative embodiment of the system of FIG. 1;

FIG. 3 shows a schematic diagram of a system for monitoring more than one utility enclosure;

FIG. 4 shows a schematic flow chart of a method for monitoring a utility enclosure;

FIG. 5 shows a schematic flow chart of a method of detecting a hot spot in a utility enclosure;

FIG. 6 shows a schematic flow chart of a method of detecting structural change in a utility enclosure;

FIG. 7 shows a schematic flow chart of a method of detecting a hazardous condition in a utility enclosure;

FIG. 8 shows a schematic flow chart of Step 760 of FIG. 7, where methane is detected as the primary reading;

FIG. 9 shows a schematic flow chart of Step 760 of FIG. 7, where Carbon Dioxide is detected as the primary reading.

### DETAILED DESCRIPTION

A number of different embodiments of the disclosure are described subsequently. In order to minimise repetition, similar features of the different embodiments are numbered with a common two-digit reference numeral and are differentiated by a third digit placed before the two common digits. Such features are structured similarly, operate similarly, and/or have similar functions unless otherwise indicated.

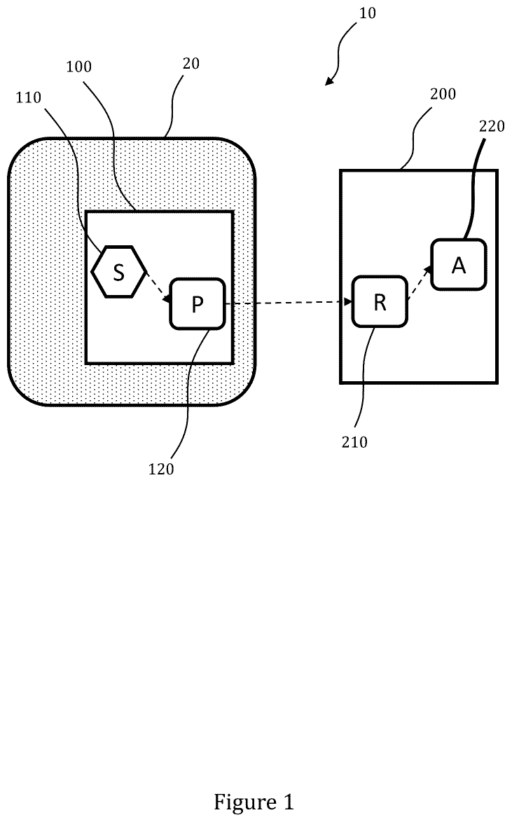

Turning now to FIG. 1, there is shown a schematic diagram of a system 10 for monitoring utility enclosures 20, or other underground utility structures such as maintenance holes, sewer holes, manholes, vaults, service boxes, or mobile vehicles. In use, the system 10 monitors the environment within the utility enclosure 20.

The system 10 comprises a sensing device 100 located within the utility enclosure 20. The sensing device 100 comprises a sensor 110 configured to detect information about the surrounding environment. As sensing device 100 is located within the utility enclosure 20, the surrounding environment is the environment within the utility enclosure 20. The sensing device 100 may comprise a plurality of sensors 110 each configured to detect different characteristics of the environment within the utility enclosure 20.

The sensing device 100 further comprises a processing module 120 configured to receive an output from the sensor 110. The sensor 110 output may be information detected by the sensor 110. The sensor 110 is configured to detect a characteristic of the environment within the utility enclosure 20.

For example, the sensor 110 may be configured to detect one or more of acetylene gas concentration, arcing presence, contact temperature, carbon monoxide gas concentration, carbon monoxide gas concentration rate of change, current, ethylene gas concentration, flooding presence, humidity, hydrogen sulphide gas concentration, pressure, methane gas concentration, methane gas concentration rate of change, fault current, salinity concentration, stray voltage presence, temperature, temperature rate of change, tert-butyl mercaptan gas concentration, and/or water level.

The processing module 120 is configured to process the sensor 110 output using one or more algorithms stored on the processing module 120. The one or more algorithms used to process the sensor 110 output are configured to determine whether the sensor 110 output is of low quality and also determine whether an anomaly exists in the detected information regarding the environment within the utility enclosure 20. The sensor 110 output is processed such that anomalies can be detected in the information output by the sensor 110.

For example, the one or more algorithms stored on the processing module 120 may determine whether the sensor 110 output comprises a blurry image, corrupt data, background noise, vertical bars in the image, and/or corrupt/erroneous pixels. The one or more algorithms stored on the processing module 120 may then improve the quality of the sensor 110 output by, for example, repairing minor errors in the images such as erroneous pixels, enhancing dark areas within the images, filtering the images to remove undesirable artifacts or contamination, and/or aligning and overlaying the original image with the processed image.

Additionally, the one of the one or more algorithms stored on the processing module 120 may determine if movement of or in the surrounding environment and/or sensing device 100 has occurred.

The system 10 further comprises an analysis system 200 located away (e.g., on a remote server) from the sensing device 100. When the one or more algorithms stored on the processing module 120 that are used to process the sensor 110 output detect an anomaly within the detected information, the processing module 120 in the sensing device 100 sends data, based on the output of the one or more algorithms, to a receiving module 210 in the analysis system 200. The data sent over from the processing module 120 to the receiving module 210 may be a processed version of the information captured by the sensor 110 on the sending device 100.

The analysis system 200 further comprises an analysis module 220 configured to receive the data from the receiving module 210 which has been sent over from the processing module 120 of the sensing device 100. The analysis module 220 is configured to process the data using one or more algorithms stored on the analysis module 220 to determine an accuracy of the determined anomaly.

The one or more algorithms stored on the processing module 120 are configured to determine whether an anomaly exists in the environment within the utility enclosure 20. The one or more algorithms stored on the processing module 120 are configured to require low computational power relative to the one or more algorithms stored on the analysis module 220. The processing module 120 has a maximum processing speed of less than 2 GHz, and a maximum SDRAM memory of less than 10 GB.

The one or more algorithms stored on the analysis module 220 are configured to confirm whether the processing module 120 accurately detected an anomaly in the environment within the utility enclosure 20. The analysis module 220 may achieve this by comparing the data sent over from the sensing device 100 which the analysis module 220 has further processed, with data stored by the analysis system 200 that was received from the sensing device 100 at an earlier time, and/or was input manually into the analysis module 220. Additionally or alternatively, the one or more algorithms stored on the analysis module 220 may confirm whether the processing module 120 accurately detected an anomaly by performing image segmentation.

The processing module 120 is configured to send data over to the analysis system 200 when an anomaly is detected and once periodically. This data sent periodically confirms that the sensing device 100 and sensor 110 are working correctly. The data sent periodically is also stored by the analysis system 200 such that comparisons can be made between data received when an anomaly is detected by the processing module and stored data at the analysis system. Such comparisons may assist in ascertaining whether anomalies exist in the future received data.

The analysis system 200 is configured to store historical data (referred to as ‘stored data’) and the one or more algorithms stored on the analysis system are configured to determine differences between the stored data and newly received data. The stored data includes previously received data, or a pre-determined condition, for example, a threshold value range. Comparing the received data with stored data allows for anomalies to be detected in the received data.

The threshold value range is configurable to a specific location's requirements. For example, the threshold value range may be broadened if the location is deemed low risk, whereas it may be tightened in areas where the risk of an anomaly could potentially be more dangerous. The threshold value range may be configured at any time.

The processing module 120 is configured to send the data to the analysis system 200 wirelessly, e.g., through a wireless connection such as a cellular connection or Wi-Fi connection. In some embodiments, the data may be sent to the analysis system 200 using a wired connection.

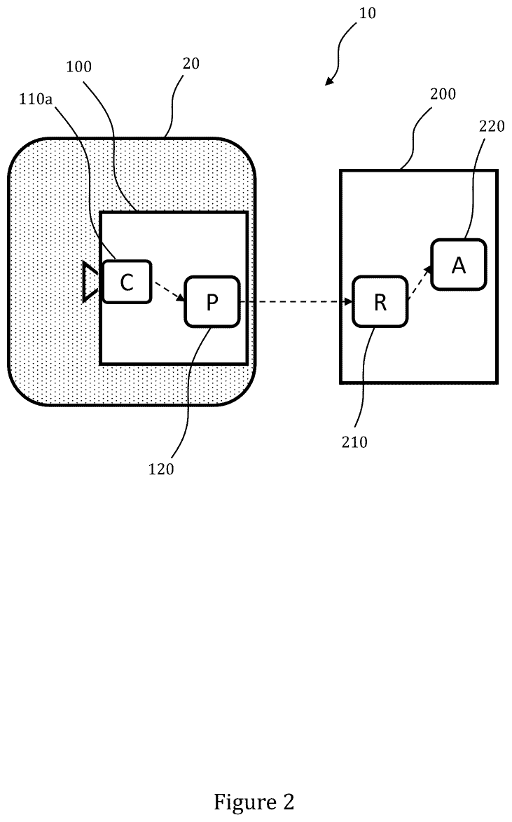

Turning now to FIG. 2, there is shown a schematic diagram of an example of the system 10 of FIG. 1. The sensor 110 is a camera 110a (i.e., a visual camera and/or infrared camera), and the output of the camera 110a comprises one or more images (e.g., a visual image and/or infrared image).

In the embodiment of FIG. 2, the one or more images are sent to the processing module 120 where they are processed and assessed for any anomalies using the one or more algorithms stored on the processing module 120. When an anomaly is detected, the processing module 120 sends the images to the receiving module 220 on the analysis system 200. The images are then sent to the analysis module 210 where they are further assessed for accuracy of the detected anomaly using the one or more algorithms stored on the analysis module 210.

As with the embodiment of FIG. 1, the processing module 120 of the embodiment of FIG. 2 also processes the data output from the sensor 110 to improve the quality of the data output and highlight any anomalies in the data output by the sensor 110. The one or more algorithms stored on the processing module 120 improve the quality of images output by the sensor/camera 110a, or modify the one or more images, for example to better show the detected anomalies.

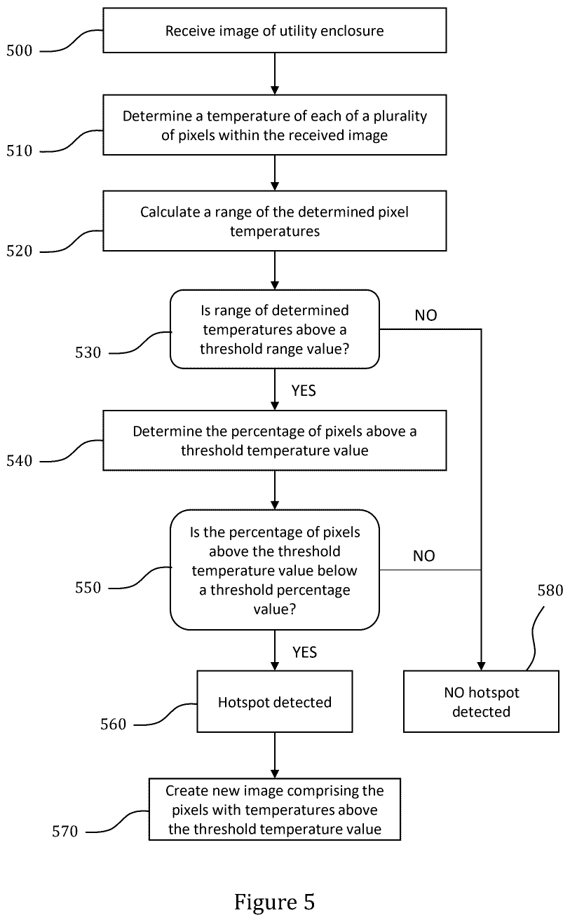

Turning now to FIG. 5, there is shown a schematic flow-chart of a method of detecting a hot spot in a utility enclosure 20. At Step 500, an image of the utility enclosure 500 is received. The image may be taken by a camera 110a on a sensing device 100 located in the utility enclosure 20.

At Step 510, the temperature of each of the plurality of pixels in the received image is determined. Each determined pixel temperature in the image of the utility enclosure represents a temperature at the respective location of the pixel within the utility enclosure 20. The image may be an infrared image. The infrared image comprises data associated with each of the plurality of pixels. The data associated with each of the plurality of pixels may include a temperature at each of the plurality of pixels.

At Step 520, a range of the determined temperatures is calculated. Each of the plurality of pixels within the image has a corresponding temperature. The range of the determined temperatures corresponds to the difference between the highest pixel temperature and the lowest pixel temperature. For example, if the highest pixel temperature is 100 degrees Celsius, and the lowest pixel temperature is 90 degrees Celsius, then the range of pixel temperatures is 10 degrees Celsius.

At Step 530, the method determines whether the range of determined pixel temperatures is above or below a temperature range threshold. If the range of determined pixel temperatures is below the threshold range temperature, then the method moves on to Step 580, whereby no hotspot is detected. If the range of temperatures is below the threshold range temperature, then it is deemed that the temperatures within the image are too close for there to be a hot spot within the utility enclosure.

However, if at Step 530, it is determined that the range of determined pixel temperatures is above the temperature range threshold then the method moves on to Step 540.

At Step 540, the percentage of pixels above a threshold temperature value is determined. The threshold temperature is at a predetermined position within the range of the determined temperatures. For example, in one embodiment of the present invention, the threshold temperature value is at a predetermined position which equates to 80% the way through the range of predetermined temperatures. For example, if the determined temperatures range from 60 degrees Celsius to 70 degrees Celsius, then the range is equivalent to 10 degrees Celsius (i.e., 70 degrees Celsius−60 degrees Celsius). Therefore, the threshold temperature value would be 60 degrees Celsius+80% of the range (i.e., 8 degrees Celsius)=68 degrees Celsius. In this particular example, the percentage of pixels above 68 degrees Celsius would be determined.

Next, at Step 550, the method determines whether the percentage of pixels above the threshold temperature value is above or below a threshold percentage value. If the percentage of pixels with temperatures above the threshold temperature value is above the threshold percentage value, then the method moves on to Step 580 whereby no hotspot is detected. However, if the percentage of pixels with temperatures above the threshold temperature is below the threshold percentage value then the method moves on to Step 560 whereby a hotspot is detected.

Next, at Step 570, the method may create and output a new image comprising the pixels with temperatures above the threshold temperature value. The new image may also comprise the pixels with temperatures equal to or below the threshold temperature value. The pixels above the threshold temperature value may be shown in one colour, and the pixels equal to or below the threshold temperature value may be shown in another colour.

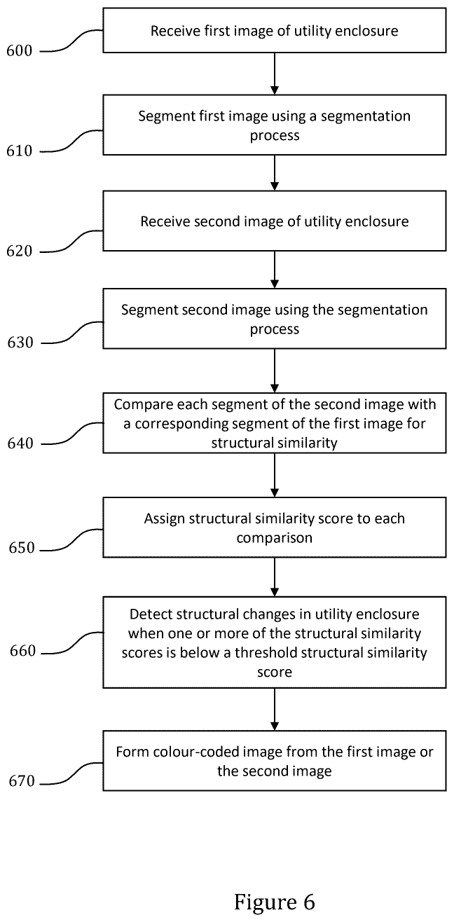

Turning now to FIG. 6, there is shown a schematic flow chart of a method of detecting a structural change in a utility enclosure 20. At Step 600, a first image of the utility enclosure 20 is received. The image may be taken by a camera 110a on a sensing device 100 located in the utility enclosure 20.

At Step 610, the first image is segmented into a plurality of first segments using a segmentation process. The segmentation process segments the first image into a plurality of first segments each comprising the same amount of pixels. The size of the segments may be selected by either a user of the method or an algorithm, optionally from a plurality of predetermined segmentation arrangements. The algorithm may select the most appropriate size of the plurality of segments for the dimensions of the first image. If it is not possible to segment the image into equally sized segments, then the method may remove one or more rows and/or columns of pixels from one or more extremities of the image, for example, the rows and columns on the right and bottom of the image.

At Step 620, a second image of the utility enclosure 20 is received. The second image may be taken by the same camera 110a as the first image.

At Step 630, the second image is segmented into a plurality of second segments using the same segmentation process as the first image. The plurality of second segments correspond in size and location to the plurality of first segments.

At Step 640, the method performs a comparison of structural similarity between corresponding segments of the first image and the second image. The comparison of structural similarity is assessed using a structural similarity algorithm. For example, structural similarity can be assessed using an algorithm that can detected changes between corresponding segments in the first image and the second image. If the structure of any component within the image was to change between the first and second image (e.g., a bend in a pipe) then the algorithm would detect the change.

At Step 650, the method assigns a structural similarity score to each of the respective comparisons between corresponding segments of the first image and the second image. The assigned structural similarity scores may be scored using a ranking system, for example, a structural similarity score of 0 may be assigned to a comparison whereby no structural similarity is noted, and, a structural similarity score of 1 may be assigned to a comparison where the two segments are deemed to be identical. A score of between 0 and 1 may be assigned to a comparison with some structural similarities and some differences.

At Step 660, the method detects a change in structural similarity if one or more of the comparisons between corresponding segments is assigned a structural similarity score below a threshold structural similarity score.

Optionally, at Step 670, the method forms a colour-coded image from the first image or the second image. At Step 650, each comparison is assigned a structural similarity score. The formed colour-coded image comprises the plurality of segments from the first/second image. Each segment is assigned a colour corresponding to the structural similarity score of the comparison. The formed image comprises the colour coding such that the final formed image displays the colour coding for each segment in a manner that is easily understood by a user of the method.

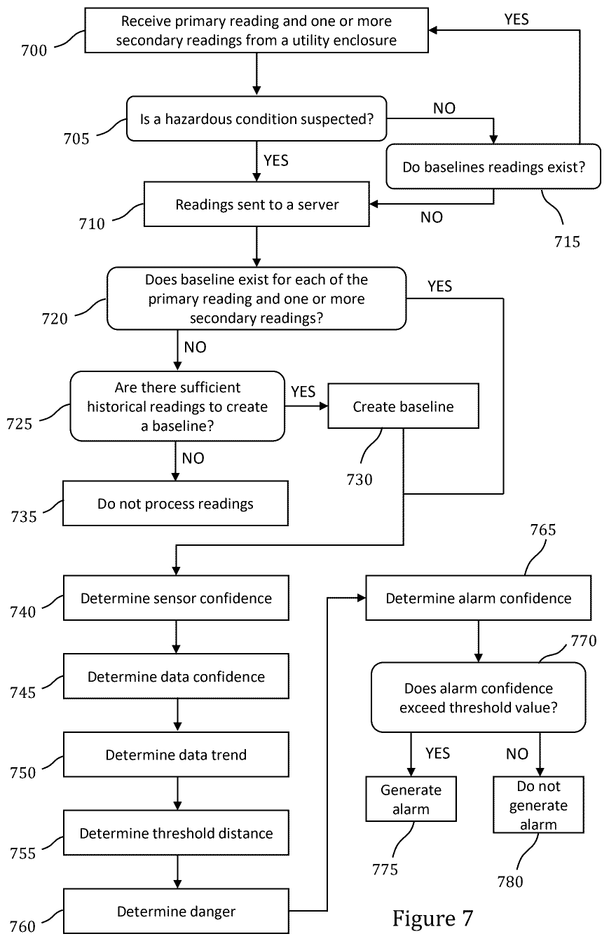

Turning now to FIG. 7, there is shown a schematic flow chart of a method of detecting hazardous conditions in a utility enclosure 20. At Step 700, a primary reading and optionally one or more secondary readings are received from the utility enclosure 20. The primary and one or more secondary readings may be taken by one or more sensors in the utility enclosure 20. The primary readings may be stray voltage, methane concentration, or carbon monoxide concentration. The secondary readings may be hot spot presence, methane concentration, carbon monoxide concentration, tert-butyl mercaptan concentration, temperature and/or stray voltage.

At Step 705, an assessment is made of whether a hazardous condition within the utility enclosure 20 is suspected. If Step 705 deems that a hazardous condition is suspected then the method sends the primary and the one or more secondary readings to a server at Step 710. If no hazardous condition is suspected then the method moves onto Step 715 where the method assesses whether baseline readings already exist. If baseline readings already exist then the method goes back to Step 700. If no baseline readings exist then the method sends the readings to the server at Step 710.

Next, at Step 720, the method assesses whether baseline readings already exist for the one or more of the primary reading and/or each of the one or more secondary readings. When baseline readings already exist for the primary reading and/or each of the one or more secondary readings, the method moves on to Step 740. Alternatively, when no baseline readings exist for one or more of the primary reading and/or the one or more secondary readings, the method moves on to Step 725 where the method assesses whether there are sufficient historical readings for the one or more of the primary reading and/or the one or more secondary readings to create the baseline readings.

When there are sufficient historical readings for the one or more of the primary reading and/or the one or more secondary readings to create the baseline readings, the method moves on to Step 730 whereby baseline readings are created for the one or more of the primary reading and/or the one or more secondary readings where no baseline readings exist.

When there are not enough historical readings, the method moves on to Step 735 whereby the primary reading and/or the one or more secondary readings are not processed, ending the method until further readings are received and the method restarts at step 700.

The baseline readings are representations of what a normal expected reading would be for each of the primary reading and/or the one or more secondary readings. The baseline readings are specific to both the device taking the readings and the location of the device. Each baseline reading consists of an expected value and one or more standard deviations. In use, the method will collect one or more of the primary readings and/or the one or more secondary readings such that baselines can be created. This means the method may require a training period such that accurate baseline readings can be generated.

Next, at Step 740, the method determines a confidence in one or more sensors that took the primary readings and/or the one or more secondary readings. Determining the confidence in the one or more sensors involves the following:

Determining if flooding has been detected where the one or more sensors are located. If flooding is detected, a sensor confidence of 0 (if the scale is 0-1) is assigned to the one or more sensors located where flooding has been detected.

Determining the humidity level where the one or more sensors are located. If the humidity level is greater than 95%, a sensor confidence of 0 (if the scale is 0-1) is assigned to the one or more sensors located where a humidity level greater than 95% has been detected. If the humidity level is between 90% and 95% then a sensor confidence of between 0-1 is assigned, whereby the sensor confidence starts at 1 (if the scale is 0-1) when humidity is at 90% and linearly decreases to 0.5 (if the scale is 0-1) when humidity is at 95%.

Determining the temperature where the one or more sensors are located. If methane is detected as the primary reading, a temperature above 50 degrees Celsius results in a sensor confidence of 0 being assigned to the one or more sensors located where a temperature greater than 50 degrees Celsius has been detected. If carbon monoxide is detected as the primary reading, a temperature above 55 degrees Celsius results in a sensor confidence of 0 being assigned to the one or more sensors. If the if the temperature is between 45 degrees Celsius and 50 degrees Celsius when methane is the primary reading then a sensor confidence of between 0-1 is assigned, whereby the sensor confidence starts at 1 (if the scale is 0-1) when the temperature is 45 degrees Celsius and linearly decreases to 0.5 (if the scale is 0-1) when the temperature is 50 degrees Celsius. If the if the temperature is between 50 degrees Celsius and 55 degrees Celsius when carbon monoxide is the primary reading then a sensor confidence of between 0-1 is assigned, whereby the sensor confidence starts at 1 (if the scale is 0-1) when the temperature is 50 degrees Celsius and linearly decreases to 0.5 (if the scale is 0-1) when the temperature is 55 degrees Celsius.

Determining the age of the one or more sensors. The methane sensor linearly loses 30% of its confidence over the first 5 years of its life and then linearly loses the remaining 70% of its confidence over the next year. The carbon monoxide sensor loses no confidence over the first 7 years of its life and then linearly loses 100% of its confidence over the next 3 years of its life. The sensor confidence for the methane sensor and the carbon monoxide sensor is adjusted based on its age.

As will be explained later, a higher sensor confidence is more likely to trigger an alarm.

Next, at Step 745, the method determines a confidence in one or more of the primary reading and/or the one or more secondary readings. This step determines whether the one or more of the primary reading and/or the one or more secondary readings are above a noise threshold. The one or more of the primary reading and/or one or more secondary readings are compared to their respective expected baseline readings. When the reading is less than two standard deviations away from the expected baseline reading, a minimum reading confidence, for example 0 when the scale is 0-1, is assigned to the reading. When the reading is more than five standard deviations away from the expected baseline reading, a maximum reading confidence of, for example 1 when the scale is 0-1, is assigned to the reading. When the reading is between two and five standard deviations away from the expected baseline reading, a reading confidence of between the minimum and maximum values, for example between 0 and 1 when the scale is 0-1, is assigned to the reading.

A weighted average is then created from the reading confidence, and, the previous one or two reading confidences. The most recent reading confidence is weighted twice that of the previous reading confidence, and the previous reading confidence is weighted twice that of the reading confidence previous to the previous reading confidence. This weighted average generates a final reading confidence.

Next, at Step 750, the method determines a trend confidence in one or more of the primary reading and/or the one or more secondary readings. This step requires a previous reading of the one or more of the primary reading and/or the one or more secondary readings. If no previous reading is available then the trend confidence is set to a minimum value, for example 0 when the scale is 0-1.

To determine a trend confidence, the number of standard deviations which the current reading and the previous reading are above or below the expected baseline reading are calculated. The difference between these two standard deviations is then calculated. A difference of less than 2 standard deviations results in a trend confidence of 0 (if the scale is 0-1). A difference of greater than 5 standard deviations results in a trend confidence of 1 (if the scale is 0-1). A difference of between 2 and 5 standard deviations results in a trend confidence of between 0 and 1 (if the scale is 0-1), whereby the trend confidence increases linearly from 0 to 1 as the number of standard deviations increases from 2 to 5.

Next, at Step 755, a distance to one or more alarm thresholds is determined. The alarm thresholds may be defined by either the method or by a user of the method. The alarm thresholds determine whether an alarm should be raised. If the one or more of the primary reading and/or one or more secondary readings is significantly above their respective alarm thresholds then a threshold confidence of close to 1 (if the scale is 0-1) will be output. If the one or more of the primary reading and one or more secondary readings is near their respective alarm thresholds then a threshold confidence of close to 0.5 (if the scale is 0-1) will be output. The determination of the threshold confidence is achieved using a Sigmoid calculation.

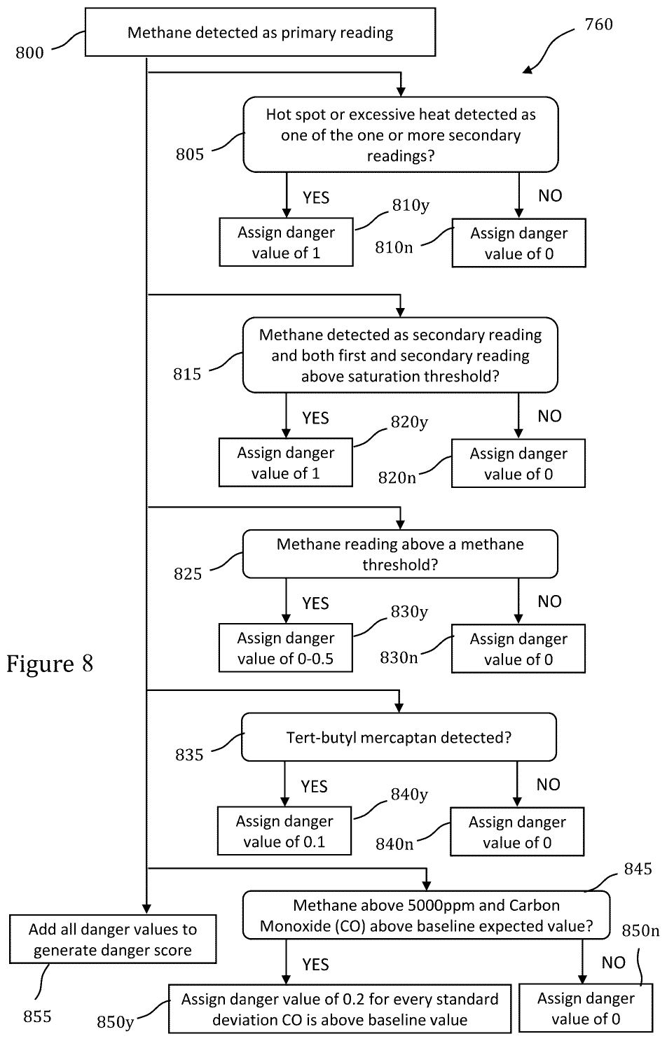

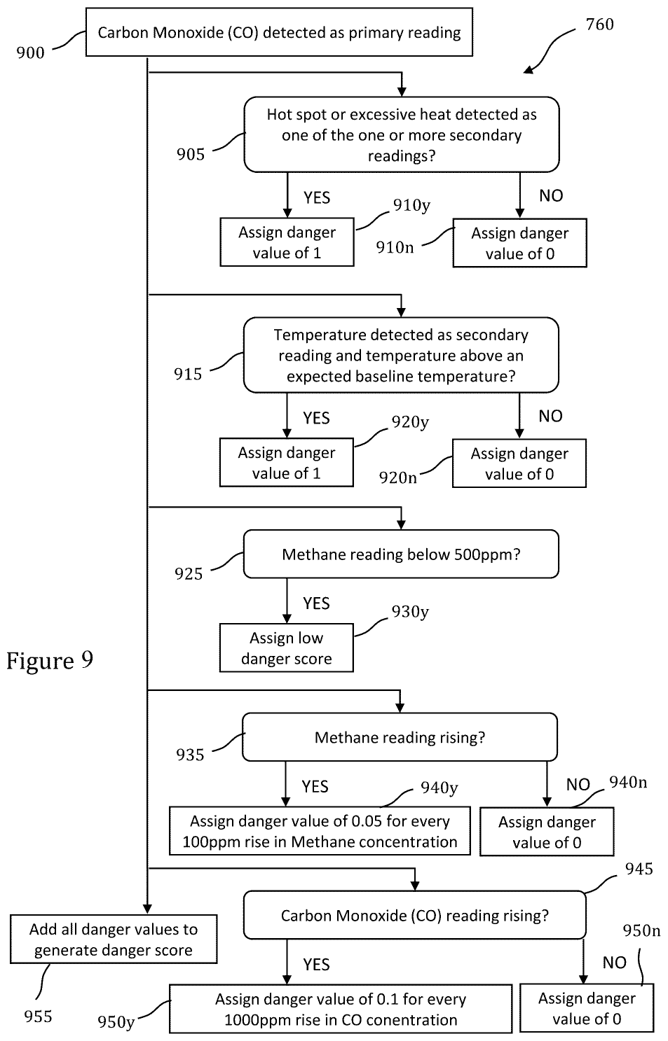

Next, at Step 760, a danger score is calculated such that a hazardous condition can be detected in the utility enclosure. This step will be explained in more detail below when describing FIGS. 8 and 9. Once Step 760 is completed, the method moves on to Step 765.

At Step 765, the alarm confidence is determined. The alarm confidence comprises two components: i) the confidence of it representing a real hazardous condition; and ii) the potential severity of the hazardous condition it is representing. The alarm confidence is determined using the sensor confidence determined in Step 740, the reading confidence determined in Step 745, the trend confidence determined in Step 750, and the threshold confidence determined in Step 755. The alarm confidence is determined by adding, or otherwise combining, these confidence values. An alarm confidence of 0 represents no confidence in the alarm, whereas an alarm confidence of over 1 represents a high degree of confidence in the alarm. An alarm confidence between 0 and 1 may represent a varying degree of confidence.

Next, at Step 770, the method determines whether the alarm confidence satisfies one or more alarm confidence threshold values. The severity of the output alarm is assessed based on the outcome of Step 760 whereby a danger score is determined. If one of the one or more alarm confidence threshold values is exceeded then the method moves on to Step 775 whereby an alarm is generated. If none of the one or more alarm confidence threshold values is exceeded then the method moves on to Step 780 whereby no alarm is generated. The severity of the alarm output in Step 775 is determined by which of the one or more alarm confidence threshold values is exceeded.

Turning now to FIG. 8, there is shown a schematic flow chart of Step 760 of FIG. 7, where methane is detected as the primary reading.

At Step 800, methane is detected as the primary reading. More specifically, a concentration of methane gas is detected as the primary reading.

Next, one or more secondary readings are taken (Step 805, and/or Step 815, and/or Step 825, and/or Step 835, and/or Step 845) within the utility enclosure. Each of the one or more secondary readings may contribute to the danger score calculated in Step 855.

At Step 805, the method determines whether a hot spot or an area of excessive heat has been detected as part of the one or more secondary readings. If a hot spot or an area of excessive heat is detected then the method assigns a danger value of 1 (if the scale is 0-1) to the detection of a hot spot or area of excessive heat (Step 810y). If no hot spot or area of excessive heat is detected then the method assigns a danger value of 0 (Step 810n).

At Step 815, the method detects a second methane reading (e.g., a second reading of a concentration of methane gas) as one of the one or more secondary readings. If both the primary reading of a concentration of methane gas and the secondary reading of a concentration of methane gas are above a saturation threshold (e.g., 9999 ppm) then, at Step 820y, the method assigns a danger value of 1 (if the scale is 0-1). If one of the methane readings is below the saturation threshold then, at Step 820n, the method assigns a danger value of 0 (if the scale is 0-1).

At Step 825, the method determines if the methane reading is above a methane alarm threshold. When the methane reading is above the methane alarm threshold value, the method, at Step 830y, assigns a danger value of between 0 and 0.5 (if the scale is 0-1) linearly dependent on where the methane reading falls between the methane alarm threshold value and the saturation threshold (e.g., 9999 ppm). When the methane reading is below the methane alarm threshold, the method, at Step 830n, will assign a danger value of 0 (if the scale is 0-1).

At Step 835, the method determines whether Tert-butyl mercaptan has been detected as one of the one or more secondary readings. When Tert-butyl mercaptan is detected, at Step 840y, the method assigns a danger value of 0.1 (if the scale is 0-1). When no tert-butyl mercaptan is detected, at Step 840n, the method assigns a danger value of 0 (if the scale is 0-1).

At Step 845, the method determines whether the detected methane concentration (primary reading) is above 5000 ppm (or any other suitable value) and whether the detected carbon monoxide concentration (one of the one or more secondary readings) is above the baseline expected value. When the methane concentration is above 5000 ppm, a danger value of 0.2 (if the scale is 0-1) is added, at Step 850y, for every one standard deviation which the concentration of carbon monoxide is above the baseline expected value. When either the methane concentration is below 5000 ppm, or, the carbon monoxide concentration is below the baseline expected value, at Step 850n, a danger value of 0 is added.

Next, at Step 855, the danger values from Steps 810, 820, 830, 840 and 850 are added together to form a danger score. The method may optionally skip anyone of Steps 805, 815, 825, 835 and/or 845 and move straight to Step 855. This may occur when any of the output danger values are greater than or equal to 1.

Turning now to FIG. 9, there is shown a schematic flow chart of Step 760 of FIG. 7, where carbon monoxide is detected as the primary reading.

At Step 900, carbon monoxide is detected as the primary reading. More specifically, a concentration of carbon monoxide gas is detected as the primary reading.

Next, one or more secondary readings are taken (Step 905, and/or Step 915, and/or Step 925, and/or Step 935, and/or Step 945) within the utility enclosure. Each of the one or more secondary readings may contribute to the danger score calculated in Step 955.

At Step 905, the method determines whether a hot spot or an area of excessive heat has been detected as part of the one or more secondary readings. If a hot spot or an area of excessive heat is detected then the method assigns a danger value of 1 (if the scale is 0-1) to the detection of a hot spot or area of excessive heat (Step 910y). If no hot spot or area of excessive heat is detected then the method assigns a danger value of 0 (Step 910n).

At Step 915, the method detects a temperature within the utility enclosure as one of the one or more secondary readings. When the detected temperature is more than two standard deviations above its baseline expected value, then, at Step 920y, the method assigns a danger value of 1 (if the scale is 0-1). However, when the detected temperature is less than two standard deviations above its baseline expected value, then, at Step 920n, the method assigns a danger value of 0 (if the scale is 0-1).

At Step 925, if methane is detected as one of the one or more secondary readings, and the methane reading is below a threshold value, for example, 500 ppm, then, at Step 930y, the method automatically assigns a low danger score (i.e., the method skips to Step 955 and assigns a low danger score).

At Step 935, the method determines whether the concentration of methane is rising. When the concentration of methane is rising (as determined by comparing more than one reading of the concentration of methane over a period of time), the method, at Step 940y, assigns a danger value of 0.05 (if the scale is 0-1) for every 100 ppm rise in the concentration of methane. When no increase in the concentration of methane is detected, then, at Step 940n, the method assigns a danger value of 0 (if the scale is 0-1).

At Step 945, the method determines whether the concentration of carbon monoxide is rising. When the concentration of carbon monoxide is rising (as determined by comparing more than one reading of the concentration of carbon monoxide over a period of time), the method, at Step 950y, assigns a danger value of 0.1 (if the scale is 0-1) for every 1000 ppm rise in the concentration of carbon monoxide. When no increase in the concentration of carbon monoxide is detected, then, at Step 950n, the method assigns a danger value of 0 (if the scale is 0-1).

Next, at Step 955, the danger values from Steps 910, 920, 930, 940 and 950 are added together to form a danger score. The method may optionally skip anyone of Steps 905, 915, 925, 935 and/or 945 and move straight to Step 955. This may occur when any of the output danger values are greater than or equal to 1.

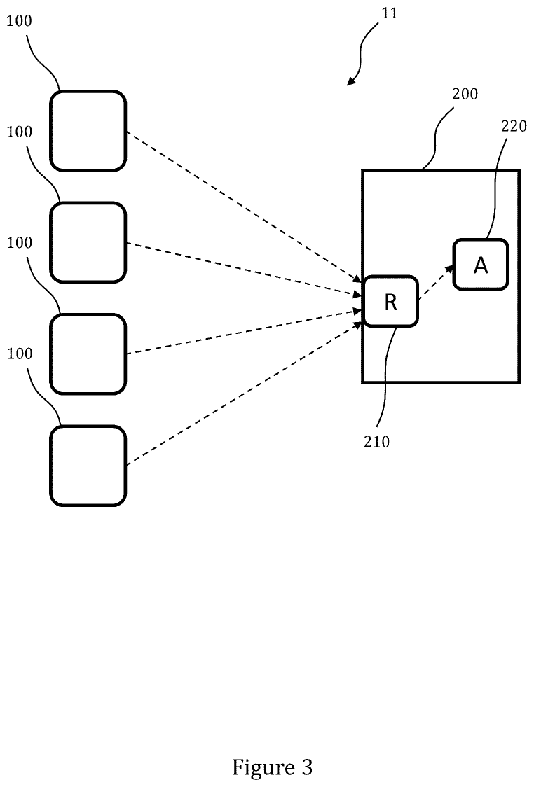

Turning now to FIG. 3, there is shown a schematic diagram of a system 11 for monitoring a plurality of different utility enclosures 20. The system 11 comprises a plurality of sensing devices 100 as described above that are each located in a different utility enclosure 20. Each of the plurality of sensing devices 100 comprises a sensor 110 and a processing module 120. Each of the sensors 110 detects information about its respective environment within its respective utility enclosure and sends data to its respective processing module 120 in the manner described above. The processing modules 120 are configured to process the data and send the data over to a common receiving module 210. The plurality of different sensing devices 100 are connected to one analysis system 200 comprising one receiving module 210 and one analysis module 220.

Alternatively, the system 11 may comprise a plurality of sensing devices 100 as described above, whereby one or more of each of the plurality of sensing devices are located in one or more of the utility enclosures 20. More than one sensing device 100 may be located within each utility enclosure 20.

Each of the plurality of sensing devices 100 are configured to wirelessly send the data to the analysis system 200. The analysis system 200 is configured to further process the anomalous images received from each of the plurality of sensing devices 100 to assess them for accuracy.

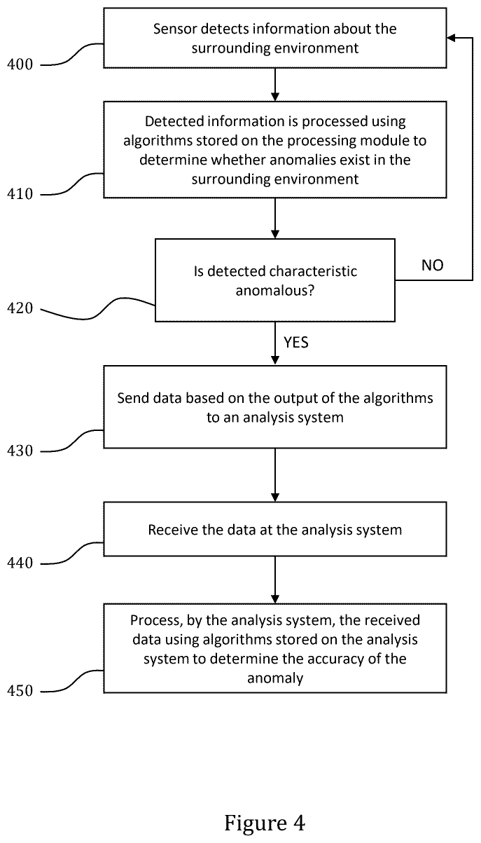

Turning now to FIG. 4, there is shown a schematic flow-chart of a method for monitoring a utility enclosure 20. At Step 400, one or more sensors 110 on the sensing device 100 located within the utility enclosure 20 detect information relating to the environment within the utility enclosure 20. The detected information may be data in the form of an image.

At Step 410, the detected information is then processed using one or more algorithms stored on the processing module 120 of the sensing device 100. The detected information is processed to improve image quality and to determine whether anomalies exist in the surrounding environment within the utility enclosure 20.

If no anomalies are detected, the method returns to Step 400 where the sensors 110 continue to detect information about the environment within the utility enclosure. At Step 430, if anomalies are detected, data based on an output of the one or more algorithms stored on the processing module 120 is then sent to the analysis system 200. The data is received at a receiving module 210 on the analysis system 200 at Step 440.

At Step 450, the received data is then sent to the analysis module 220 within the analysis system 200, whereby the data is processed using one or more algorithms stored on the analysis system 200 to determine whether the anomalies were accurately detected.

Once an anomaly is confirmed by the analysis module 220, an alert is sent to a user.

Although particular embodiments of the disclosure have been disclosed herein in detail, this has been done by way of example and for the purposes of illustration only. The aforementioned embodiments are not intended to be limiting with respect to the scope of the summary/appended claims.

It is contemplated by the inventors that various substitutions, alterations, and modifications may be made to the invention without departing from the scope of the invention as defined by the claims.

The following statements describe aspects of the invention:

Statement 1. A computer-implemented method of detecting a hot spot in a utility enclosure, the method comprising:
receiving an infrared image of the utility enclosure, wherein the infrared image comprises a plurality of pixels;
determining a temperature of each of the plurality of pixels within the infrared image;
calculating a range of the determined temperatures, wherein the range of the determined temperatures is a difference between a highest temperature of the determined temperatures and a lowest temperature of the determined temperatures;
assessing whether the range of the determined temperatures is above a threshold range value;
when the range of the determined temperatures is above the threshold range value, determining a percentage of the plurality of pixels with temperatures above a threshold temperature value, wherein the threshold temperature value is a temperature between the highest temperature and the lowest temperature; and
detecting the hot spot when the percentage of the plurality of pixels with temperatures above the threshold temperature value is below a threshold percentage of the plurality pixels.

Statement 2. The method according to statement 1, the method further comprising: creating, from the infrared image, a new image comprising the pixels with determined temperatures above the threshold temperature value, when more than or equal to one and less than all of the determined temperatures are above the threshold temperature value.

Statement 3. The method of statement 1 or 2, wherein the threshold temperature value is between 60 degrees Celsius and 70 degrees Celsius.

Statement 4. The method according to any one of statements 1 to 3, wherein the threshold range value is between 4 degrees Celsius and 8 degrees Celsius.

Statement 5. The method according to any one of statements 1 to 4, wherein the threshold percentage of the plurality of pixels is between 0.5% and 2%.

Statement 6. A computer-implemented method of detecting a structural change in a utility enclosure, the method comprising:
receiving a first image of the utility enclosure;
dividing the first image into a first plurality of segments using an image segmentation process; storing the first image comprising the plurality of segments;
receiving a second image of the utility enclosure and dividing the second image into a second plurality of segments using the image segmentation process;
comparing each segment of the second image with a corresponding one of the segments of the first image, wherein comparing comprises assessing for structural similarity;
assigning, based on the assessment of structural similarity, a structural similarity score to each comparison; and
detecting a structural change in the utility enclosure when the structural similarity score of one or more of the comparisons is below a threshold structural similarity score.

Statement 7. The method of statement 6 further comprising the step of forming a colour-coded image from the first image or the second image, wherein the colour-coded image comprises the first or second plurality of segments, and wherein each of the plurality of segments is assigned a colour code corresponding to the structural similarity score of the respective comparison.

Statement 8. The method of statement 6 or 7, wherein each of the plurality of segments comprises a common number of pixels.

Statement 9. A computer-implemented method of detecting a hazardous condition in a utility enclosure, the method comprising:
receiving a primary reading comprising a concentration of a first gas in the utility enclosure;
receiving one or more secondary readings taken in the utility enclosure; and
detecting a hazardous condition based on the primary reading and the one or more secondary readings.

Statement 10. The method of statement 9, the method further comprising:
determining a danger value based on the primary reading and the one or more secondary readings; and
detecting a hazardous condition when the danger value is above a threshold danger value.

Statement 11. The method of statement 10, wherein the first gas is methane.

Statement 12. The method of statement 10, wherein the first gas is carbon monoxide.

Statement 13. The method of statement 11 or 12, wherein one of the one or more secondary readings is a hot spot determined by any one of statements 1 to 5.

Statement 14. The method of statement 11 or 13 when dependent on statement 11, wherein one of the one or more secondary readings is a concentration of carbon monoxide.

Statement 15. The method of statement 12 or 13 when dependent on statement 12, wherein one of the one or more secondary readings is a concentration of methane.

Statement 16. The method of statement 12 or 15 or statement 13 when dependent on statement 12, wherein one of the one or more secondary readings is a temperature.

Statement 17. The method of any one of statement 11, 14 or statement 13 when dependent on statement 11, wherein one of the one or more secondary readings is a positive detection of tert-butyl mercaptan.

Statement 18. The method of any one of statements 9 to 17, wherein a danger score is assigned to one or more of the primary reading and to the one or more secondary readings, and wherein the danger value is determined from the one or more danger scores.

1. A system for monitoring one or more utility enclosures, the system comprising:
a sensing device, and
an analysis system,
wherein the sensing device comprises:
a sensor configured to detect information about a surrounding environment, and
a processing module configured to:
receive sensor output from the sensor,
process the sensor output using one or more algorithms stored on the processing module to determine whether an anomaly exists in the surrounding environment, and
send data based on output of the one or more algorithms to the analysis system, and wherein the analysis system comprises:
a receiving module configured to receive the data from the sensing device, and
an analysis module configured to process the received data using one or more algorithms stored on the analysis system to determine accuracy of the determined anomaly.

2. A system according to claim 1, wherein the processing module is configured to perform the step of sending data to the analysis system when the anomaly is determined.

3. A system according to claim 1 or claim 2, wherein the processing module is configured to send the data to the analysis system wirelessly.

4. A system according to any preceding claim, wherein the data comprises an indication of the determined anomaly.

5. A system according to any preceding claim, wherein the sensor is a camera and the output of the sensor is one or more images.

6. A system according to claim 5, wherein the sensor is a visual and/or infrared camera.

7. A system according to any preceding claim, wherein the data comprises:
a text description, and/or
one or more images, and
optionally, tags assigned to the one or more images.

8. A system according to any preceding claim wherein the one or more algorithms stored on the processing module of the sensing device have a lower computational requirement than the one or more algorithms stored on the analysis system.

9. A system according to claim 8, wherein the processing module of the sensing device has a maximum processing speed of less than 2 GHz.

10. A system according to any one of claims 8 or 9, wherein the processing module of the sensing device has a maximum SDRAM memory of less than 10 GB.

11. A system according to any preceding claim wherein the one or more algorithms stored on the processing module comprise an algorithm operable on sensor output to determine if the sensor output is of low quality, and/or improve the quality of the sensor output.

12. A system according to any preceding claim wherein the one or more algorithms stored on the analysis system comprise an algorithm operable to determine differences between the received data and stored data, wherein the stored data is data stored by the analysis system and received from the sensing device at a time before the received data.

13. A sensing device for a utility enclosure, the sensing device comprising:
a sensor configured to detect information about a surrounding environment, and
a processing module configured to:
receive sensor output from the sensor,
process the sensor output using one or more algorithms stored on the processing module to determine whether an anomaly exists in the surrounding environment, and
send data based on output of the one or more algorithms to an analysis system.

14. A sensing device according to claim 13, wherein the processing module is configured to perform the step of sending data to the analysis system when the anomaly is determined.

15. A sensing device according to claim 13 or 14, wherein the sensor is a camera and the output of the sensor is one or more images and the one or more algorithms stored on the processing module comprise an algorithm operable on each of the sensor output image(s) to determine if each image is of low quality, and/or improve the quality of the sensor output.

16. An analysis system for monitoring utility enclosures, the analysis system comprising:
a receiving module configured to receive data from a sensing device located at a utility enclosure, and
an analysis module configured to process the received data using one or more algorithms stored on the analysis system to determine an accuracy of an anomaly of the utility enclosure indicated by the received data.

17. An analysis system according to claim 16, wherein the data comprises an indication of the determined anomaly.

18. An analysis system according to claim 16 or 17, wherein the received data comprises on or more images and the one or more algorithms stored on the analysis system comprise image segmentation.

19. An analysis system according to claim 16, 17 or 18, wherein the one or more algorithms stored on the analysis system comprise an algorithm operable to determine differences between the received data and stored data, wherein the stored data is data stored by the analysis system and received from the sensing device at a time before the received data.

20. A method of monitoring a utility enclosure, the method comprising:
detecting, within the utility enclosure, information about the utility enclosure,
processing, within the utility enclosure, the detected information using one or more algorithms to determine whether an anomaly exists in the environment, and
sending data based on output of the one or more algorithms to an analysis system outside of the utility enclosure,
receiving the data at the analysis system, and
further processing the received data, by the analysis system, using one or more algorithms stored on the analysis system to determine accuracy of the determined anomaly.

21. A method according to claim 20, wherein the step of sending data to the analysis system is performed when the anomaly is determined.

22. A method according to claim 20 or 21, wherein the one or more algorithms performed within the utility enclosure have a lower computational requirement that the one or more algorithms performed by the analysis system.

23. A method according to any of claims 20 to 22, wherein the one or more algorithms performed within the utility enclosure comprise an algorithm operable on detected information to determine if the detected information is of low quality, and/or improve the quality of the detected information.

24. A method according to any of claims 20 to 23, wherein the one or more algorithms performed by the analysis system comprise an algorithm operable to determine differences between the received data and stored data, wherein the stored data is data stored by the analysis system and received from within the utility enclosure at a time before the received data.

25. A computer-readable storage medium storing instructions adapted to carry out the method described above of monitoring a utility enclosure.
